(19) United States
(12) Patent Application Publication
(10) Pub. No.: US 2007/0134339 A1
(43) Pub. Date: Jun. 14, 2007

(54) ZONISAMIDE AND NSAID NANOPARTICULATE FORMULATIONS

(75) Inventors: Scott Jenkins, Downingtown, PA (US); Gary G. Liversidge, West Chester, PA (US)

Correspondence Address: FOLEY AND LARDNER LLP;SUITE 500, 3000 K STREET NW, WASHINGTON, DC, 20007, US

(73) Assignee: Elan Pharma International, Limited

(21) Appl. No.: 11/469,315

(22) Filed: Aug. 31, 2006

Related U.S. Application Data
(60) Provisional application No. 60/712,920, filed on Aug. 31, 2005.

(51) Int. Cl. A61K 31/42; A61K 9/14

(52) U.S. Cl. 424/489; 514/379; 977/906

(57) ABSTRACT

The present invention is directed to compositions comprising zonisamide, or a salt or derivative thereof, and at least one nanoparticulate NSAID. The zonisamide and NSAID combination preferably includes nanoparticulate NSAID particles of the composition with an effective average particle size of less than about 2000 nm. The zonisamide and NSAID combination is useful in the treatment of migraine and acute migraine pain and related conditions.

### CROSS-REFERENCE TO RELATED PATENT APPLICATION

This application claims the benefit under 35 U.S.C. § 119(e) to U.S. Provisional Application No. 60/712,920, filed on Aug. 31, 2005, which is incorporated herein in its entirety by reference.

### FIELD OF INVENTION

The present invention relates generally to compounds and compositions useful in the treatment of migraine and acute migraine pain and related conditions. More specifically, the invention relates to zonisamide and NSAID combination compositions, particularly combinations having one or more nanoparticulate NSAIDS. The nanoparticulate NSAID components of the combination compositions, when present, have an effective average particle size of less than about 2000 nm.

### BACKGROUND OF INVENTION

A. Background Regarding Zonisamide

Zonisamide, chemically known as 1,2-benzisoxazole-3-methanesulfonamide or 3-sulfamoylmethyl-1,2-benzisoxazole, is an antiseizure drug. Zonisamide, CAS no. 68291-97-4, has an empirical formula of C₈H₈N₂O₃S, and molecular weight of 212.23. The chemical structure of zonisamide is shown below:

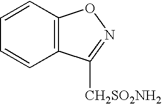

Zonisamide is a white powder, having a pKa=10.2, and is moderately soluble in water (0.80 mg/mL) and 0.1 N HCl (0.50 mg/mL). Zonisamide is commercially available in the United States under the trade name of ZONEGRAN®. ZONEGRAN® is supplied for oral administration as a two-piece hard gelatin capsules containing 100 mg zonisamide and the inactive ingredients of microcrystalline cellulose, hydrogenated vegetable oil, sodium laurel sulfate, gelatin, and colorants. ZONEGRAN® capsules have a white opaque body and red opaque cap, with company logo and “ZONEGRAN 100” in printed in black. ZONEGRAN® is distributed by Elan Biopharmaceuticals, a business unit of Elan Pharmaceuticals, Inc., San Diego, Calif.; ZONEGRAN® is a trademark licensed exclusively to Elan Pharmaceuticals, Inc.

ZONEGRAN® is generally administered once or twice daily, except for the daily dose of 100 mg at the initiation of therapy. After two weeks, the dose may be increased to 200 mg/day for at least two weeks. It can be increased to 300 mg/day and 400 mg/day, with the dose stable for at least two weeks to achieve steady state at each level. Because of the long half-life of zonisamide, up to two weeks may be required to achieve steady state levels upon reaching a stable dose or following dosage adjustment. ZONEGRAN® is given orally and can be taken with or without food. Following a 200-400 mg oral zonisamide dose, peak plasma concentrations (range: 2-5 μg/mL) in normal volunteers occur within 2-6 hours. In the presence of food, the time to maximum concentration is delayed, occurring at 4-6 hours, but food has no effect on the bioavailability of zonisamide.

Zonisamide is indicated as adjunctive therapy in the treatment of partial seizures in adults with epilepsy. Although Zonisamide has demonstrated anticonvulsant activity in several experimental models, the precise mechanism(s) by which zonisamide exerts its antiseizure effect is unknown.

Zonisamide compounds, and derivatives thereof, have been described in, for example, U.S. Pat. No. 4,172,896 to Ueno et al. for “Methane-Sulfonamide Derivatives, the Preparation Thereof and Compositions Comprising the Same”, U.S. Pat. No. 6,342,515 to Masuda et al for “Remedy for Neurodegenerative Diseases”, U.S. Pat. No. 6,489,350 to Benedyk for “Methods for Treating Neuropathic Pain Using Heteroarylmethanesulfonamides”, U.S. Pat. No. 6,841,683 to Mendelovici et al. for “Sulfonation Method for Zonisamide Intermediate in Zonisamide Synthesis and Their Novel Crystal Forms”, U.S. Pat. No. 6,900,333 to Ueno et al. for “Process for the Preparation of 1,2-Dichlorethane Free Crystals of Zonisamide”, U.S. Patent Application No. 2003/0036556 to Jennings for “Zonisamide Use in Headache”, U.S. Patent Application No. 2004/0029941 to Jennings for “Zonisamide Use in Obesity and Eating Disorders”, U.S. Patent Application No. 2005/0026977 to Jennings for “Zonisamide Use in Eating Disorders”, U.S. Patent Application No. 2005/0043704 to Lieberburg for “Methods of Using Zonisamide as an Adjunctive Therapy for Partial Seizures”, and WO2004024096 to Shellenberger for “Method of Treating Tremors”.

B. Background Regarding NSAIDS

Non-steroidal anti-inflammatory drugs, usually abbreviated “NSAIDS”, are drugs with analgesic, antipyretic and anti-inflammatory effects, i.e., NSAIDS reduce pain, fever and inflammation. These activities are derived from the common mechanism of the inhibition of cyclooxygenase, which is the critical enzyme for the biosynthesis of prostaglandins, prostacyclin, and thromboxanes. Because prostaglandins are released in response to inflammatory stimuli, which in turn result in inflammatory responses (e.g., redness, pain, heat and swelling of tissue), inhibition of prostaglandins by NSAIDS results in analgesia. The NSAIDS include the categories of propionic acid derivatives; acetic acid derivatives; fenamic acid derivatives; biphenylcarboxylic acid derivatives; and oxicams. Additionally, NSAIDS can be broadly classified based on their chemical structure. NSAIDS within a group will tend to have similar characteristics and tolerability. There is little difference in clinical efficacy between the NSAIDS when used at equivalent doses. Differences between compounds tended to be with regards to dosing regimens (related to half-life), route of administration, and tolerability profile. For example, salicylates include aspirin, methyl salicylate and diflunisal, arylalkanoic acids include indomethacin, sulindac and diclofenac; 2-arylpropionic acids (profens) include ibuprofen, ketoprofen, naproxen and ketorolac; N-arylanthranilic acids (fenamic acids) include mefenamic acid; oxicams include piroxicam and meloxicam; coxibs include celecoxib, rofecoxib (withdrawn from market), valdecoxib, parecoxib and etoricoxib; and sulphonanilides include nimesulide. Most NSAIDS act as non-selective inhibitors of the enzyme cyclooxygenase, i.e., they inhibit both the cyclooxygenase-1 (COX-1) and cyclooxygenase-2 (COX-2) isoenzymes. Cyclooxygenase catalyses the formation of prostaglandins and thromboxane from arachidonic acid (itself derived from the cellular phospholipid bilayer by phospholipase A₂). Prostaglandins act (among other things) as messenger molecules in the process of inflammation.

NSAIDS are usually indicated for the treatment of acute or chronic conditions where pain and inflammation are present. In 2001, NSAIDS accounted for 70,000,000 prescriptions and 30 billion over-the-counter doses are sold annually in the United States. As a result, NSAIDS are well-known for treatment of minor discomfort and illnesses such as cold, aches and pains, mild fever, osteoarthritis, rheumatoid arthritis, acute or severe pain, etc. In the central nervous system, NSAIDS are anti-hyperanalgesic through a direct action on the spinal cord. NSAIDS, as used herein, include any non-narcotic analgesic nonsteroidal anti-inflammatory compound, including pharmaceutically acceptable salts thereof which fall within the classes of compounds set forth above. The acceptable salts include sodium, potassium, arginine, lysine, and the like. The term “non-steroidal” is used to distinguish these drugs from steroids. NSAIDS are sometimes also referred to as non-steroidal anti-inflammatory agents/analgesics (NSAIAs).

The dosage of the NSAID will vary according to the potency of the specific compound. Therapeutic doses for these analgesics are well known in the art and can be found in the Physician's Desk Reference (Medical Economics Company, Montvale, N.J.). For example, the preferred dosage of NSAID is 40-800 milligrams of ibuprofen every 4 to 6 hours.

Representative NSAIDS of the present invention are described in U.S. Pat. No. 4,985,459 to Sunshine et al., issued Jan. 15, 1991, incorporated by reference herein. Preferred propionic NSAIDS include, for example, aspirin, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Specific examples of the propioinic acid derivatives include ibuprofen, naproxen, naproxen sodium, fenoprefen, ketoprofen and the like. Suitable acetic acid derivatives include indomethacin, zomepirac, sulindac and the like. Suitable fenamic acid derivatives include mefenamic acid and meclofenamate sodium. Suitable biphenylcarboxylic acid derivatives include diflunisal and flufenisal. Combinations of such NSAIDS are also contemplated in the present invention. Some of the most well known NSAIDS are ibuprofen, ketoprofen, naproxen and aspirin.

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto, or associated with, the surface thereof a non-crosslinked surface stabilizer. The '684 patent does not describe compositions of zonisamide and nanoparticulate NSAID combinations.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” 6,432,381 for “Methods for Targeting Drug Delivery to the Upper and/or Lower Gastrointestinal Tract,” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” 6,656,504 for “Nanoparticulate Compositions Comprising Amorphous Cyclosporine;” 6,742,734 for “System and Method for Milling Materials;” 6,745,962 for “Small Scale Mill and Method Thereof;” 6,811,767 for “Liquid Droplet Aerosols of Nanoparticulate Drugs;” 6,908,626 for “Compositions Having a Combination of Immediate Release and Controlled Release Characteristics;” 6,969,529 for “Nanoparticulate Compositions Comprising Copolymers of Vinyl Pyrrolidone and Vinyl Acetate as Surface Stabilizers;” 6,976,647 for “System and Method for Milling Materials;” and 6,991,191 for “Method of Using a Small Scale Mill;” all of which are specifically incorporated by reference.

In addition, U.S. Patent Publication No. 20020012675 A1, for “Controlled Release Nanoparticulate Compositions;” U.S. Patent Publication No. 20060188566 for “Nanoparticulate formulations of docetaxel and analogues thereof”; U.S. Patent Publication No. 20060165806 for “Nanoparticulate candesartan formulations”; U.S. Patent Publication No. 20060159767 for “Nanoparticulate bicalutamide formulations”; U.S. Patent Publication No. 20060159766 for “Nanoparticulate tacrolimus formulations”; U.S. Patent Publication No. 20060159628 for “Nanoparticulate benzothiophene formulations”; U.S. Patent Publication No. 20060154918 for “Injectable nanoparticulate olanzapine formulations”; U.S. Patent Publication No. 20060121112 for “Topiramate pharmaceutical composition”; U.S. Patent Publication No. 20050276974 for “Nanoparticulate Fibrate Formulations;” U.S. Patent Publication No. 20050238725 for “Nanoparticulate Compositions Having a Peptide as a Surface Stabilizer;” U.S. Patent Publication No. 20050233001 for “Nanoparticulate Megestrol Formulations;” U.S. Patent Publication No. 20050147664 for “Compositions Comprising Antibodies and Methods of Using the Same for Targeting Nanoparticulate Active Agent Delivery;” U.S. Patent Publication No. 20050063913 for “Novel Metaxalone Compositions;” U.S. Patent Publication No. 20050042177 for “Novel Compositions of Sildenafil Free Base;” U.S. Patent Publication No. 20050031691 for “Gel Stabilized Nanoparticulate Active Agent Compositions;” U.S. Patent Publication No. 20050019412 for “Novel Glipizide Compositions;” U.S. Patent Publication No. 20050004049 for “Novel Griseofulvin Compositions;” U.S. Patent Publication No. 20040258758 for “Nanoparticulate Topiramate Formulations;” U.S. Patent Publication No. 20040258757 for “Liquid Dosage Compositions of Stable Nanoparticulate Active Agents;” U.S. Patent Publication No. 20040229038 for “Nanoparticulate Meloxicam Formulations;” U.S. Patent Publication No. 20040208833 for “Novel Fluticasone Formulations;” U.S. Patent Publication No. 20040195413 for “Compositions and Method for Milling Materials;” U.S. Patent Publication No. 20040156895 for “Solid Dosage Forms Comprising Pullulan;” U.S. Patent Publication No. U.S. Patent Publication No. U.S. Patent Publication No. 20040156872 for “Novel Nimesulide Compositions;” U.S. Patent Publication No. 20040141925 for “Novel Triamcinolone Compositions;” U.S. Patent Publication No. 20040115134 for “Novel Nifedipine Compositions;” U.S. Patent Publication No. 20040105889 for “Low Viscosity Liquid Dosage Forms;” U.S. Patent Publication No. 20040105778 for “Gamma Irradiation of Solid Nanoparticulate Active Agents;” U.S. Patent Publication No. 20040101566 for “Novel benzoyl peroxide compositions;” U.S. Patent Publication No. 20040057905 for “Nanoparticulate Beclomethasone Dipropionate Compositions;” U.S. Patent Publication No. 20040033267 for “Nanoparticulate Compositions of Angiogenesis Inhibitors;” U.S. Patent Publication No. 20040033202 for “Nanoparticulate Sterol Formulations and Novel Sterol Combinations;” U.S. Patent Publication No. 20040018242 for “Nanoparticulate nystatin formulations;” U.S. Patent Publication No. 20040015134 for “Drug delivery Systems and Methods;” U.S. Patent Publication No. 20030232796 for “Nanoparticulate Polycosanol Formulations & Novel Polycosanol Combinations;” U.S. Patent Publication No. 20030215502 for “Fast Dissolving Dosage Forms Having Reduced Friability;” U.S. Patent Publication No. 20030185869 for “Nanoparticulate Compositions Having Lysozyme as a Surface Stabilizer;” U.S. Patent Publication No. 20030181411 for “Nanoparticulate Compositions of Mitogen-Activated Protein (MAP) Kinase Inhibitors;” U.S. Patent Publication No. 20030137067 for “Compositions Having a Combination of Immediate Release and Controlled Release Characteristics;” U.S. Patent Publication No. 20030108616 for “Nanoparticulate Compositions Comprising Copolymers of Vinyl Pyrrolidone and Vinyl Acetate as Surface Stabilizers;” U.S. Patent Publication No. 20030095928 for “Nanoparticulate Insulin;” U.S. Patent Publication No. 20030087308 for “Method for High Through-put Screening Using a Small Scale Mill or Microfluidics;” U.S. Patent Publication No. 20030023203 for “Drug Delivery Systems & Methods;” U.S. Patent Publication No. 20020179758 for “System and Method for Milling Materials;” and U.S. Patent Publication No. 20010053664 for “Apparatus for Sanitary Wet Milling,” describe nanoparticulate active agent compositions and are specifically incorporated by reference. None of these references describe compositions of zonisamide and nanoparticulate NSAID combinations.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

### SUMMARY OF THE INVENTION

The present invention relates to zonisamide and NSAID combination compositions, particularly combination compositions that comprise at least one nanoparticulate NSAID. The combination compositions comprise zonisamide or a salt or derivative thereof, at least one nanoparticulate NSAID, and at least one surface stabilizer adsorbed on or associated with the surface of the NSAID particles. The nanoparticulate NSAID particles have an effective average particle size of less than about 2,000 nm.

Additionally, the compositions may comprise at least one primary and at least one secondary surface stabilizer. Exemplary surface stabilizers may include one or more of an anionic surface stabilizer, a cationic surface stabilizer, a non-ionic surface stabilizer, a zwitterionic surface stabilizers, and an ionic surface stabilizer.

The compositions described herein may be formulated for dosage or administration in a variety of forms. Although any pharmaceutically acceptable dosage form may be utilized, dosage forms contemplated include, but are not limited to formulations for oral, pulmonary, rectal, colonic, parenteral, intracistemal, intravaginal, intraperitoneal, ocular, otic, local, buccal, nasal, topical, liquid dispersions, gels, aerosols, ointments, creams, bioadhesives, lyophilized formulations, tablets, capsules, controlled release formulations, fast melt formulations, delayed release formulations, extended release formulations, pulsatile release formulations, mixed immediate release, controlled release formulations and combinations thereof. In some embodiments, solid dosages, such as an oral tablet, may be preferred.

Another aspect of the invention is directed to pharmaceutical compositions comprising zonisamide or a salt or derivative thereof, at least one nanoparticulate NSAID, at least one surface stabilizer, and a pharmaceutically acceptable carrier, as well as any desired excipients.

The compositions disclosed herein are also contemplated to exhibit improved pharmacokinetic properties as compared to compositions comprising a non-nanoparticulate form of the same NSAID.

One embodiment of the invention encompasses a composition comprising zonisamide, or a salt or derivative thereof, and at least one nanoparticulate NSAID, wherein the pharmacokinetic profile of the zonisamide and nanoparticulate NSAID composition is not affected by the fed or fasted state of a subject ingesting the composition.

In yet another embodiment, the invention encompasses a composition comprising zonisamide, or a salt or derivative thereof, and at least one nanoparticulate NSAID, wherein administration of the composition to a subject in a fasted state is bioequivalent to administration of the composition to a subject in a fed state.

Another embodiment of the invention is directed to a composition comprising zonisamide, or a salt or derivative thereof, and at least one nanoparticulate NSAID, and further comprising one or more additional compounds useful in the treatment of migraine and acute migraine pain and related conditions.

This invention further discloses a method of making the inventive zonisamide and nanoparticulate NSAID combination compositions. Such a method comprises contacting at least one NSAID with at least one surface stabilizer for a time and under conditions sufficient to provide a nanoparticulate NSAID having an effective average particle size of less than about 2000 nm, followed by combining the nanoparticulate NSAID with zonisamide, or a salt or derivative thereof, to produce the desired combination composition. In some methods, contacting may include grinding, wet grinding, homogenization, freezing, template emulsion, precipitation, supercritical fluid particle generation techniques and combinations thereof.

The present invention is also directed to methods of treatment including but not limited to, the treatment of migraine and acute migraine pain and related conditions, using the novel zonisamide and nanoparticulate NSAID combination compositions disclosed herein. Such methods comprise administering to a subject a therapeutically effective amount of a composition comprising zonisamide, or a salt or derivative thereof, and at least one nanoparticulate NSAID.

Both the foregoing summary and the following the detailed description are exemplary and explanatory and are intended to provide further details of the compositions and methods as claimed. Other objects, advantages, and novel features will be readily apparent to those skilled in the art from the following detailed description.

### DETAILED DESCRIPTION OF THE INVENTION

The present invention provides a combination zonisamide and NSAID therapy effective in the treatment and prevention of migraine, particularly for the treatment of acute migraine pain. The present invention is directed to the treatment of migraine pain, particularly acute migraine pain, using a combination dosage formulation comprising zonisamide, or a salt or derivative thereof, and one or more nanoparticulate NSAIDS. In addition, the present invention is further directed to a combination dosage form having a dosage package in which separate doses of zonisamide, or a salt or derivative thereof, and one or more nanoparticulate NSAID dosages are packaged together in one patient convenient dosing package.

Furthermore, the combination zonisamide and nanoparticulate NSAID composition of the present invention is preferably formulated with at least one nanoparticulate NSAID useful for a rapid onset of therapeutic effect of the NSAID for immediate treatment of migraine pain. Representative nanoparticulate NSAID components include, for example without limitation, propionic-, propionic acid derivative-, acetic acid derivative-, fenamic acid derivative-biphenylcarboxylic-, acid derivative-NSAIDS and combinations thereof. In some embodiments, preferred NSAIDS may include ketoprofen, ibuprofen, meloxicam, naproxen, and combinations thereof, effective for the treatment of migraine and related conditions. The nanoparticulate NSAID component benefits from enhanced dissolution rates, because of the small particle size of the drug, resulting in increased bioavailability and therefore enhanced rates of drug absorption. This also provides a counter to the gastric stasis often associated with acute migraine pain. The at least one nanoparticulate NSAID may be combined with zonisamide, or a salt or derivative thereof, into a single dosage form or packaged together in a single dosage package.

I. Zonisamide and NSAID Combination Compositions

The present invention is directed to compositions comprising zonisamide, or a salt or derivative thereof, and at least one nanoparticulate NSAID. Preferably, the compositions also comprise at least one surface stabilizer which is adsorbed on, or associated with, the surface of the NSAID particles. The nanoparticulate NSAID particles preferably have an effective average particle size of less than about 2000 nm.

Advantages of the compositions of the invention, comprising zonisamide, or a salt or derivative thereof, and at least one nanoparticulate NSAID, include, but are not limited to: (1) smaller tablet or other solid dosage form size; (2) smaller doses of NSAID required to obtain the same pharmacological effect as compared to conventional microcrystalline forms of the same NSAID; (3) increased bioavailability for the NSAID as compared to conventional microcrystalline forms of the same NSAID; (4) substantially similar pharmacokinetic profiles of the nanoparticulate NSAID component when administered in the fed versus the fasted state; (5) bioequivalency of the NSAID component when administered in the fed versus the fasted state; (6) an increased rate of dissolution for the NSAID component as compared to conventional microcrystalline forms of the same NSAID; and (7) the zonisamide and NSAID combination compositions can be used in conjunction with other active agents useful in the treatment of migraine and acute migraine pain and related conditions.

The present invention also compositions comprising zonisamide, or a salt or derivative thereof, and at least one nanoparticulate NSAID, together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, ocular, local (powders, ointments, or drops), buccal, intracisternal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

A. Definitions

The present invention is described herein using several definitions, as set forth below and throughout the application.

The term “effective average particle size of less than about 2000 nm,” as used herein, means that at least about 50% of the nanoparticulate NSAID particles have a size of less than about 2000 nm (by weight or by other suitable measurement technique, such as by number or by volume) when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable nanoparticulate NSAID particles, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase, (3) that the particles are chemically stable; and/or (4) where the NSAID has not been subject to a heating step at or above the melting point of the NSAID in the preparation of the nanoparticles of the present invention,

The term “conventional” or “non-nanoparticulate” active agent shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixtures thereof irrespective of their size, shape or morphology.

B. Preferred Characteristics of the Zonisamide and NSAID Combination Compositions of the Invention

1. Increased Bioavailability

The compositions of the invention, comprising zonisamide, or a salt or derivative thereof, and at least one nanoparticulate NSAID, are proposed to exhibit increased bioavailability of the NSAID component, and require smaller doses as compared to prior conventional non-nanoparticulate NSAID formulations.

2. Improved Pk Profiles for the Nanoparticulate NSAID

The NSAID component of the compositions described herein may also exhibit a desirable pharmacokinetic profile when administered to mammalian subjects. The desirable pharmacokinetic profile of the NSAID component preferably includes, but is not limited to: (1) a Cₘₐₓ for the NSAID, when assayed in the plasma of a mammalian subject following administration, that is preferably greater than the Cₘₐₓ for a non-nanoparticulate formulation of the same NSAID, administered at the same dosage; and/or (2) an AUC for the NSAID, when assayed in the plasma of a mammalian subject following administration, that is preferably greater than the AUC for a non-nanoparticulate formulation of the same NSAID, administered at the same dosage; and/or (3) a Tₘₐₓ for the NSAID, when assayed in the plasma of a mammalian subject following administration, that is preferably less than the Tₘₐₓ for a non-nanoparticulate formulation of the same NSAID, administered at the same dosage The desirable pharmacokinetic profile, as used herein, is the pharmacokinetic profile measured after the initial dose of the NSAID.

In one embodiment, the nanoparticulate NSAID exhibits in comparative pharmacokinetic testing with a non-nanoparticulate formulation of the same NSAID, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate NSAID formulation.

In another embodiment, the nanoparticulate NSAID exhibits in comparative pharmacokinetic testing with a non-nanoparticulate formulation of the same NSAID, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate NSAID,

In yet another embodiment, the nanoparticulate NSAID exhibits in comparative pharmacokinetic testing with a non-nanoparticulate formulation of the same NSAID, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate NSAID.

3. The Pharmacokinetic Profiles of the Zonisamide and Nanoparticulate NSAID Combination Compositions of the Invention are not Affected by the Fed or Fasted State of the Subject Ingesting the Compositions

The invention encompasses zonisamide and nanoparticulate NSAID combination compositions wherein the pharmacokinetic profile of the nanoparticulate NSAID is not substantially affected by the fed or fasted state of a subject ingesting the composition. This means that there is no substantial difference in the quantity of NSAID absorbed or the rate of NSAID absorption when the zonisamide and nanoparticulate NSAID combination compositions are administered in the fed versus the fasted state.

For conventional NSAID formulations, the absorption of the NSAID is increased when administered with food. This difference in absorption observed with conventional NSAID formulations is undesirable. The zonisamide and nanoparticulate NSAID combination formulations of the invention overcome this problem, as the zonisamide and nanoparticulate NSAID combination formulations reduce or preferably substantially eliminate significantly different absorption levels when administered under fed as compared to fasting conditions for the NSAID.

The difference in absorption of the nanoparticulate NSAID compositions, when administered in the fed versus the fasted state, preferably is less than about 100%, less than about 90%, less than about 80%, less than about 70%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

Benefits of a dosage form which substantially eliminates the effect of food include an increase in subject convenience, thereby increasing subject compliance, as the subject does not need to ensure that they are taking a dose either with or without food.

4. Bioequivalency of Zonisamide and Nanoparticulate NSAID Combination Compositions of the Invention When Administered in the Fed Versus the Fasted State

The invention also provides a zonisamide and nanoparticulate NSAID combination composition in which administration of the composition to a subject in a fasted state is bioequivalent to administration of the composition to a subject in a fed state.

In some embodiments, the invention encompasses compositions wherein administration of the NSAID component to a subject in a fasted state is bioequivalent to administration of the NSAID component to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Zonisamide and Nanoparticulate NSAID Combination Compositions of the Invention

The compositions of the invention, comprising zonisamide, or a salt or derivative thereof, and at least one nanoparticulate NSAID, are proposed to have unexpectedly dramatic dissolution profiles. Rapid dissolution of an administered active agent is preferable, as faster dissolution generally leads to faster onset of action and greater bioavailability. To improve the dissolution profile and bioavailability of the NSAID it would be useful to increase the drug's dissolution so that it could attain a level close to 100%.

The nanoparticulate NSAID component of the compositions of the invention preferably has a dissolution profile in which within about 5 minutes at least about 20% of the composition is dissolved. In other embodiments of the invention, at least about 30% or at least about 40% of the nanoparticulate NSAID component is dissolved within about 5 minutes. In yet other embodiments of the invention, preferably at least about 40%, at least about 50%, at least about 60%, at least about 70%, or at least about 80% of the nanoparticulate NSAID component is dissolved within about 10 minutes. Finally, in another embodiment of the invention, preferably at least about 70%, at least about 80%, at least about 90%, or at least about 100% of the nanoparticulate NSAID component is dissolved within 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersability of the Zonisamide and Nanoparticulate NSAID Combination Compositions of the Invention

An additional feature of the compositions of the invention, comprising zonisamide, or a salt or derivative thereof, and at least one nanoparticulate NSAID, is that the nanoparticulate NSAID particles redisperse such that the effective average particle size of the redispersed NSAID particles is less than about 2 microns. This is significant, as if upon administration the nanoparticulate NSAID particles did not redisperse to a substantially nanoparticulate size, then the dosage form may lose the benefits afforded by formulating the NSAID into a nanoparticulate size.

This is because nanoparticulate active agent compositions benefit from the small particle size of the active agent; if the active agent does not disperse into the small particle sizes upon administration, them “clumps” or agglomerated active agent particles are formed, owing to the extremely high surface free energy of the nanoparticulate system and the thermodynamic driving force to achieve an overall reduction in free energy. With the formulation of such agglomerated particles, the bioavailability of the dosage form my fall well below that observed with the liquid dispersion form of the nanoparticulate active agent.

Moreover, the nanoparticulate NSAID component of the compositions of the invention exhibits dramatic redispersion of the nanoparticulate NSAID particles upon administration to a mammal, such as a human or animal, as demonstrated by reconstitution/redispersion in a biorelevant aqueous media such that the effective average particle size of the redispersed NSAID particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, water, aqueous electrolyte solutions or aqueous solutions of any salt, acid, or base, or a combination thereof, which exhibit the desired pH and ionic strength. Such redispersion in a biorelevant media is predictive of in vivo efficacy of the NSAID dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed NSAID particles of the invention (redispersed in a biorelevant media, an aqueous media, or any other suitable media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Zonisamide and Nanoparticulate NSAID Combination Compositions Used in Conjunction with Other Active Agents

The compositions of the invention comprising zonisamide, or a salt or derivative thereof, and at least one nanoparticulate NSAID, can additionally comprise one or more compounds useful in the treatment of migraine and acute migraine pain and related conditions, or the compositions of the invention can be administered in conjunction with such a compound. Such compounds include, but are not limited to narcotic analgesics, such as, but not limited to, morphine, codeine, hydrocodone, and oxycodone.

C. Zonisamide and Nanoparticulate NSAID Combination Compositions

The invention provides compositions comprising zonisamide, or a salt or derivative thereof, at least one nanoparticulate NSAID, and preferably at least one surface stabilizer. The surface stabilizers preferably are adsorbed on, or associated with, the surface of the nanoparticulate NSAID particles. Surface stabilizers especially useful herein preferably physically adhere on, or associate with, the surface of the nanoparticulate NSAID particles, but do not chemically react with the nanoparticulate NSAID particles or itself, Individually adsorbed molecules of the surface stabilizer are essentially free of intermolecular cross-linkages.

The present invention also includes zonisamide, or a salt or derivative thereof, and at least one nanoparticulate NSAID, together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

1. Zonisamide

The compositions of the invention comprise zonisamide or a salt or derivative thereof. The zonisamide can be in crystalline phase, semi-crystalline phase, amorphous phase, semi-amorphous phase, or a combination thereof.

2. NSAID Particles

The compositions of the invention comprise at least one nanoparticulate NSAID. The NSAID particles can be in crystalline phase, semi-crystalline phase, amorphous phase, semi-amorphous phase, or a combination thereof.

Exemplary NSAIDS include, but are not limited to, the categories of propionic acid derivatives; acetic acid derivatives; fenamic acid derivatives; biphenylcarboxylic acid derivatives; and oxicams. Salicylates include aspirin, methyl salicylate and diflunisal; arylalkanoic acids include indomethacin, sulindac and diclofenac; 2-arylpropionic acids (profens) include ibuprofen, ketoprofen, naproxen and ketorolac; N-arylanthranilic acids (fenamic acids) include mefenamic acid; oxicams include piroxicam and meloxicam; coxibs include celecoxib, rofecoxib, valdecoxib, parecoxib and etoricoxib; and sulphonanilides include nimesulide.

Exemplary propionic NSAIDS include, for example, aspirin, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Specific examples of the propioinic acid derivatives include ibuprofen, naproxen, naproxen sodium, fenoprefen, ketoprofen and the like. Suitable acetic acid derivatives include indomethacin, zomepirac, sulindac and the like. Suitable fenamic acid derivatives include mefenamic acid and meclofenamate sodium. Suitable biphenylcarboxylic acid derivatives include diflunisal and flufenisal. Some of the most well known NSAIDS are ibuprofen, ketoprofen, naproxen and aspirin.

Other examples of NSAIDS include, but are not limited to, deiflunisal, zomepirac sodium, ibuprofen, naproxen, fenoprofen, piroxicam, flurbiprofen, mefenamic acid and sulindac.

3. Surface Stabilizers

The choice of a surface stabilizer for a zonisamide and nanoparticulate NSAID combination is non-trivial and required extensive experimentation to realize a desirable formulation. Accordingly, the present invention is directed to the surprising discovery that zonisamide and nanoparticulate NSAID combination compositions can be made.

Combinations of more than one surface stabilizers can be used in the invention. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, ionic, anionic, cationic, and zwitterionic surfactants or compounds.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethylammonium chloride, trimethylammonium halide, alkyl-trimethyl ammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylam onium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁—R₄ are CH₃;
(ii) one of R₁—R₄ is CH₃;
(iii) three of R₁—R₄ are CH₃;
(iv) all of R₁—R₄ are CH₃;
(v) two of R₁—R₄ are CH₃, one of R₁—R₄ is C₆H₅CH₂, and one of R₁—R₄ is an alkyl chain of seven carbon atoms or less;
(vi) two of R₁—R₄ are CH₃, one of R₁—R₄ is C₆H₅CH₂, and one of R₁—R₄ is an alkyl chain of nineteen carbon atoms or more;
(vii) two of R₁—R₄ are CH₃ and one of R₁—R₄ is the group C₆H₅(CH₂)ₙ, where n>1;
(viii) two of R₁—R₄ are CH₃, one of R₁—R₄ is C₆H₅CH₂, and one of R₁—R₄ comprises at least one heteroatom;
(ix) two of R₁—R₄ are CH₃, one of R₁—R₄ is C₆H₅CH₂, and one of R₁—R₄ comprises at least one halogen;
(x) two of R₁—R₄ are CH₃, one of R₁—R₄ is C₆H₅CH₂, and one of R₁—R₄ comprises at least one cyclic fragment;
(xi) two of R₁—R₄ are CH₃ and one of R₁—R₄ is a phenyl ring; or
(xii) two of R₁—R₄ are CH₃ and two of R₁—R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

4. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

5. NSAID Particle Size

The compositions of the invention comprise particles of at least one NSAID which have an effective average particle size of less than about 2000 nm (i.e., 2 microns), less than about 1900 nm, less than about 1800 nm, less than about 1700 nm, less than about 1600 mm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 n” it is meant that at least 50% of the NSAID combination particles have a particle size of less than the effective average, by weight (or by other suitable measurement technique, such as by volume, number, etc.), i.e., less than about 2000 nm, 1900 nm, 1800 nm, etc., when measured by the above-noted techniques. In other embodiments of the invention, at least about 60%, at least about 70%, at least about 80%, at least about 90%, at least about 95%, or at least about 99% of the NSAID particles have a particle size of less than the effective average, i.e., less than about 2000 mm, 1900 nm, 1800 nm, 1700 nm, etc.

In the present invention, the value for D50 of a nanoparticulate NSAID component is the particle size below which 50% of the NSAID particles fall, by weight (or by other suitable measurement technique, such as by volume, number, etc.). Similarly, D90 is the particle size below which 90% of the NSAID particles fall, by weight (or by other suitable measurement technique, such as by volume, number, etc.).

6. Concentration of Zonisamide and Nanoparticulate NSAID Combination and Surface Stabilizers

The relative amounts of zonisamide, or a salt or derivative thereof, at least one nanoparticulate NSAID, and at least one surface stabilizer can vary widely. The optimal amount of the individual components can depend, for example, upon the particular zonisamide and nanoparticulate NSAID combination selected, the hydrophilic lipophilic balance (HLB), melting point, and the surface tension of water solutions of the stabilizer, etc.

The concentration of the at least one nanoparticulate NSAID can vary from about 99.5% to about 0.001%, from about 95% to about 0.1%, or from about 90% to about 0.5%, by weight, based on the total combined dry weight of the nanoparticulate NSAID and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the nanoparticulate NSAID and at least one surface stabilizer, not including other excipients.

The amount of zonisamide present in a composition of the invention may vary, and may range from about 1 milligram (“mg”) to about 500 mg per dose, from about 10 mg to about 250 mg per dose, and from about 25 mg to about 100 mg per dose.

The ratio of zonisamide to NSAID may also vary, and may range from about 1 part zonisamide to about 100 parts NSAID per dose and from about 100 parts zonisamide to about 1 part NSAID per dose.

7. Exemplary Zonisamide and Nanoparticulate NSAID Combination Tablet Formulations

Several exemplary zonisamide and nanoparticulate NSAID combination tablet formulations are given below. These examples are not intended to limit the claims in any respect, but rather to provide exemplary tablet formulations of zonisamide and nanoparticulate NSAID combination which can be utilized in the methods of the invention. Such exemplary tablets can also comprise a coating agent.

Exemplary Zonisamide and Nanoparticulate NSAID
Combination Tablet Formulation #1

| Component | g/Kg |
|---|---|
| Zonisamide and NSAID | about 50 to about 500, each |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Zonisamide and Nanoparticulate NSAID
Combination Tablet Formulation #2

| Component | g/Kg |
|---|---|
| Zonisamide and NSAID | about 100 to about 300, each |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Zonisamide Nanoparticulate NSAID
Combination Tablet Formulation #3

| Component | g/Kg |
|---|---|
| Zonisamide and NSAID | about 200 to about 225, each |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Zonisamide and Nanoparticulate NSAID
Combination Tablet Formulation #4

| Component | g/Kg |
|---|---|
| Zonisamide and NSAID | about 119 to about 224, each |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

D. Methods of Making Zonisamide and Nanoparticulate NSAID Combination Compositions

The composition of the invention, comprising zonisamide, or a salt or derivative thereof, and at least one nanoparticulate NSAID, can be made using, for example, milling, homogenization, precipitation, freezing, or template emulsion techniques. Exemplary methods of making nanoparticulate active agent compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable DrugNanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant zonisamide and nanoparticulate NSAID combination compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Zonisamide and Nanoparticulate NSAID Combination Dispersions

Milling an NSAID to obtain a nanoparticulate NSAID dispersion comprises dispersing particles of an NSAID in a liquid dispersion medium in which the NSAID is poorly soluble, followed by applying mechanical means in the presence of grinding media to reduce the particle size of the NSAID to the desired effective average particle size, e.g., less than about 2000 Mn. The dispersion medium can be, for example, water, safflower oil, ethanol, t-butanol, glycerin, polyethylene glycol (PEG), hexane, or glycol. A preferred dispersion medium is water.

The NSAID particles can be reduced in size in the presence of at least one surface stabilizer. Alternatively, NSAID particles can be contacted with one or more surface stabilizers after attrition. Other compounds, such as a diluent, can be added to the NSAID/surface stabilizer composition during the size reduction process. Dispersions can be manufactured continuously or in a batch mode.

Following particle size reduction of the NSAID, zonisamide, or a salt or derivative thereof, can be added to form a combination composition according to the invention.

2. Precipitation to Obtain Zonisamide and Nanoparticulate NSAID Combination Compositions

Another method of forming the desired composition of the invention, comprising zonisamide, or a salt or derivative thereof, and at least one nanoparticulate NSAID, is by microprecipitation of the NSAID. This is a method of preparing stable dispersions of poorly soluble active agents in the presence of one or more surface stabilizers and one or more colloid stability enhancing surface active agents free of any trace toxic solvents or solubilized heavy metal impurities, Such a method comprises, for example: (1) dissolving the NSAID in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

Following particle size reduction of the NSAID, zonisamide, or a salt or derivative thereof, can be added to form a combination composition according to the invention.

3. Homogenization to Obtain Zonisamide and Nanoparticulate NSAID Combination Compositions

Exemplary homogenization methods of preparing nanoparticulate active agent compositions are described in U.S. Pat. No. 5,510,118, for “Process of Preparing Therapeutic Compositions Containing Nanoparticles.” Such a method comprises dispersing particles of an NSAID in a liquid dispersion medium, followed by subjecting the dispersion to homogenization to reduce the particle size of a NSAID to the desired effective average particle size. The NSAID particles can be reduced in size in the presence of at least one surface stabilizer. Alternatively, the NSAID particles can be contacted with one or more surface stabilizers either before or after attrition. Other compounds, such as a diluent, can be added to the NSAID/surface stabilizer composition either before, during, or after the size reduction process. Dispersions can be manufactured continuously or in a batch mode,

Following particle size reduction of the NSAID, zonisamide, or a salt or derivative thereof, can be added to form a combination composition according to the invention.

4. Cryogenic Methodologies to Obtain Zonisamide and Nanoparticulate NSAID Combination Compositions

Another method of forming the desired nanoparticulate NSAID, or a salt or derivative thereof, composition is by spray freezing into liquid (SFL). This technology comprises an organic or organoaqueous solution of NSAID with stabilizers, which is injected into a cryogenic liquid, such as liquid nitrogen. The droplets of the NSAID solution freeze at a rate sufficient to minimize crystallization and particle growth, thus formulating nanostructured NSAID particles. Depending on the choice of solvent system and processing conditions, the NSAID particles can have varying particle morphology. In the isolation step, the nitrogen and solvent are removed under conditions that avoid agglomeration or ripening of the NSAID particles.

As a complementary technology to SFL, ultra rapid freezing (URF) may also be used to created equivalent nanostructured NSAID particles with greatly enhanced surface area, URF comprises an organic or organoaqueous solution of NSAID with stabilizers onto a cryogenic substrate.

Following particle size reduction of the NSAID, zonisamide, or a salt or derivative thereof, can be added to form a combination composition according to the invention.

5. Emulsion Methodologies to Obtain Zonisamide and Nanoparticulate NSAID Combination Compositions

Another method of forming the desired composition of the invention, comprising zonisamide, or a salt or derivative thereof, and at least one nanoparticulate NSAID, is by template emulsion. Template emulsion creates nanostructured NSAID particles with controlled particle size distribution and rapid dissolution performance. The method comprises an oil-in-water emulsion that is prepared, then swelled with a non-aqueous solution comprising the NSAID and stabilizers. The particle size distribution of the NSAID particles is a direct result of the size of the emulsion droplets prior to loading with the NSAID a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured NSAID particles are recovered. Various NSAID particles morphologies can be achieved by appropriate control of processing conditions.

Following particle size reduction of the NSAID, zonisamide, or a salt or derivative thereof, can be added to form a combination composition according to the invention.

E. Methods of Using the Zonisamide and Nanoparticulate NSAID Combination Compositions of the Invention

The invention provides a method of increasing bioavailability of a zonisamide and nanoparticulate NSAID combination, or a salt or derivative thereof, in a subject. Such a method comprises orally administering to a subject an effective amount of a composition comprising a zonisamide and NSAID combination. The zonisamide and NSAID combination composition, in accordance with standard pharmacokinetic practice, has a bioavailability that is about 50% greater than a conventional dosage form, about 40% greater, about 30% greater, about 20% or about 10% greater.

The compositions of the invention are useful in the treatment of migraine and acute migraine pain and related conditions.

The zonisamide and nanoparticulate NSAID combination, or a salt or derivative thereof, compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The zonisamide and nanoparticulate NSAID combination, or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a zonisamide and NSAID combination, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

‘Therapeutically effective amount’ as used herein with respect to a zonisamide and nanoparticulate NSAID combination, dosage shall mean that dosage that provides the specific pharmacological response for which a zonisamide and nanoparticulate NSAID combination is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that zonisamide and nanoparticulate NSAID combination dosages are, in particular instances, measured as oral dosages, or with reference to drug levels as measured in blood.

One of ordinary skill will appreciate that effective amounts of a zonisamide and nanoparticulate NSAID combination can be determined empirically and can be employed in pure form or, where such forms exist, in pharmaceutically acceptable salt, ester, or prodrug form. Actual dosage levels of a zonisamide and nanoparticulate NSAID combination in the nanoparticulate compositions of the invention may be varied to obtain an amount of a zonisamide and nanoparticulate NSAID combination that is effective to obtain a desired therapeutic response for a particular composition and method of administration. The selected dosage level therefore depends upon the desired therapeutic effect, the route of administration, the potency of the administered zonisamide and nanoparticulate NSAID combination, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### II. EXAMPLES

The purpose of this prophetic example is to describe how a composition comprising zonisamide, or a salt or derivative thereof, and at least one nanoparticulate NSAID, can be made.

An aqueous dispersion of 5% (w/w) of an NSAID, combined with one or more surface stabilizers, such as hydroxypropyl cellulose (HPC-SL) and dioctylsulfosuccinate (DOSS), could be milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical Co.) (e.g., at an 89% media load). In an exemplary process, the mixture could be milled at a speed of 2500 rpm for 60 minutes.

Following milling, the particle size of the milled NSAID particles can be measured, in deionized distilled water, using a Horiba LA 910 particle size analyzer. For a successful composition, the initial mean and/or D50 milled NSAID particle size is expected to be less than 2000 nm.

The nanoparticulate NSAID composition is then combined with zonisamide, or a salt or derivative thereof, to form a composition according to the invention.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A composition comprising:
(a) zonisamide or a salt or derivative thereof;
(b) particles of at least one NSAID having an average effective particle size of less than about 2000 nm; and
(c) at least one surface stabilizer.

2. The composition of claim 1, wherein zonisamide or at least one NSAID is in a crystalline phase, an amorphous phase, a semi-crystalline phase, a semi amorphous phase, or a mixture thereof.

3. The composition of claim 1, wherein the effective average particle size of the NSAID particles is selected from the group consisting of less than about 1900 n, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 n, less than about 75 nm, and less than about 50 nm.

4. The composition of claim 1, wherein the composition is formulated:
(a) for administration selected from the group consisting of oral, pulmonary, intravenous, rectal, ophthalmic, colonic, parenteral, intracistemal, intravaginal, intraperitoneal, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, mixed immediate release formulations, controlled release formulations; or
(d) any combination of (a), (b), and (c).

5. The composition of claim 1, wherein the composition further comprises one or more pharmaceutically acceptable excipients, carriers, or a combination thereof.

6. The composition of claim 1, additionally comprising one or more active agents useful for the treatment of acute migraine pain and related conditions.

7. The composition of claim 6, wherein said one or more active agents is selected from the group consisting of morphine, codeine, hydrocodone, oxycodone, and combinations thereof.

8. The composition of claim 1, wherein
(a) the amount of the NSAID is selected from the group consisting of from about 99.5% to about 0.001%, from about 95% to about 0.1%, and from about 90% to about 0.5%, by weight, based on the total combined dry weight of the NSAID and at least one surface stabilizer, not including other excipients;
(b) at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the NSAID and at least one surface stabilizer, not including other excipients, or
(c) a combination of (a) and (b).

9. The composition of claim 1, further comprising at least one primary surface stabilizer and at least one secondary surface stabilizer.

10. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

11. The composition of claim 1, wherein at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate (dioctyl sodium sulfosuccinate), dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

12. The composition of claim 1, wherein the pharmacokinetic profile of the NSAID is not significantly affected by the fed or fasted state of a subject ingesting the composition.

13. The composition of claim 1, wherein the NSAID component does not produce significantly different absorption levels when administered under fed as compared to fasting conditions.

14. The composition of claim 13, wherein the difference in absorption of the NSAID component of the composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

15. The composition of claim 1, wherein administration of the NSAID component of the composition to a subject in a fasted state is bioequivalent to administration of said composition to a subject in a fed state.

16. The composition of claim 15, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

17. The composition of claim 1, wherein:
(a) the Tₘₐₓ of the NSAID, when assayed in the plasma of a mammalian subject following administration, is less than the Tₘₐₓ for a non-nanoparticulate composition of the same NSAID, administered at the same dosage;
(b) the Cₘₐₓ of the NSAID, when assayed in the plasma of a mammalian subject following administration, is greater than the Cₘₐₓ for a non-nanoparticulate composition of the same NSAID, administered at the same dosage;
(c) the AUC of the NSAID, when assayed in the plasma of a mammalian subject following administration, is greater than the AUC for a non-nanoparticulate composition of the same NSAID, administered at the same dosage; or
(d) any combination of (a), (b), and (c).

18. The composition of claim 17, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same NSAID, administered at the same dosage;
(b) the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same NSAID, administered at the same dosage;
(c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the same NSAID, administered at the same dosage; or
(d) any combination of (a), (b), and (c).

19. The composition claim 1, wherein:
(a) upon administration to a mammal the NSAID particles redisperse such that the particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1110 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm;
(b) the composition redisperses in a biorelevant media such that the NSAID particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 mm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm; or
(c) a combination of (a) and (b).

20. The composition of claim 19, wherein the biorelevant media is selected from the group consisting of water, aqueous electrolyte solutions, aqueous solutions of a salt, aqueous solutions of an acid, aqueous solutions of a base, and combinations thereof.

21. A method of preparing a zonisamide and nanoparticulate NSAID combination composition comprising:
(a) contacting particles of at least one NSAID with at least one surface stabilizer for a time and under conditions sufficient to provide a nanoparticulate NSAID composition having an effective average particle size of less than about 2000 nm; and
(b) combining the nanoparticulate NSAID composition with zonisamide or a salt or derivative thereof.

22. The method of claim 21, wherein the contacting comprises grinding, wet grinding, homogenization, freezing, emulsion techniques, supercritical fluid particle generation techniques, precipitation, or a combination thereof.

23. A method for the treatment of acute migrane pain and related conditions in a subject comprising administering to a subject of an effective amount of a composition comprising:
(a) zonisamide or a salt or derivative thereof;
(b) particles of at least one NSAID having an average effective particle size of less than about 2000 nm; and
(c) at least one surface stabilizer.

24. The method of claim 23, wherein at least one NSAID is selected from the group consisting of a propionic NSAID, a propionic acid derivative NSAID, an acetic acid derivative NSAID, a fenamic acid derivative NSAID, a biphenylcarboxylic NSAID, and an acid derivative NSAID.

25. The method of claim 23, wherein at least one NSAID is selected from the group consisting of aspirin, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, naproxen sodium, indomethacin, zomepirac, sulindac, mefenamic acid, meclofenamate sodium, diflunisal, flufenisal, methyl salicyate, diflunisal, diclofenac, ketorolac, piroxicam, meloxicaim, celecoxib, rofecoxib, valdecoxib, paracoxib, etoricoxib and nimesulide.

26. The method of claim 23, wherein at least one NSAID is selected from the group consisting of ketoprofen, ibuprofen, meloxicam, naproxen and asprin.

27. The method of claim 23, further comprising one or more active agents useful for the treatment of acute migraine pain and related conditions.

28. The method of claim 27, wherein at least one of the or more active agents is selected from the group consisting of morphine, codeine, hydrocodone, oxycodone, and combinations thereof.

29. The method of claim 23, wherein the composition is in the form of an oral tablet.
